(19) United States
(12) Patent Application Publication
(10) Pub. No.: US 2007/0004771 A1
(43) Pub. Date: Jan. 4, 2007

(54) Preparation of 1,6,7-trisubstituted azabenzimidazoles as kinase inhibitors

(75) Inventors: Dennis Lee, King of Prussia, PA (US); Robert A. Stravenger, King of Prussia, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(73) Assignee: Glaxo Group Limited

(21) Appl. No.: 10/574,652

(22) PCT Filed: Oct. 6, 2004
(86) PCT No.: PCT/US04/32909

§ 371 Date: Apr. 4, 2006

Related U.S. Application Data
(60) Provisional application No. 60/509,123, filed on Oct. 6, 2003.

(51) Int. Cl. C07D 471/02; A61K 31/4745

(52) U.S. Cl. 514/303; 546/117

(57) ABSTRACT

Novel inhibitors of Rho-kinases are disclosed.

An important large family of enzymes is the protein kinase enzyme family. Currently, there are about 500 different known protein kinases. Protein kinases serve to catalyze the phosphorylation of an amino acid side chain in various proteins by the transfer of the γ-phosphate of the ATP-Mg²⁺ complex to said amino acid side chain. These enzymes control the majority of the signaling processes inside cells, thereby governing cell function, growth, differentiation and destruction (apoptosis) through reversible phosphorylation of the hydroxyl groups of serine, threonine and tyrosine residues in proteins. Studies have shown that protein kinases are key regulators of many cell functions, including signal transduction, transcriptional regulation, cell motility, and cell division. Several oncogenes have also been shown to encode protein kinases, suggesting that kinases play a role in oncogenesis. These processes are highly regulated, often by complex intermeshed pathways where each kinase will itself be regulated by one or more kinases. Consequently, aberrant or inappropriate protein kinase activity can contribute to the rise of disease states associated with such aberrant kinase activity. Due to their physiological relevance, variety and ubiquitousness, protein kinases have become one of the most important and widely studied family of enzymes in biochemical and medical research.

The protein kinase family of enzymes is typically classified into two main subfamilies: Protein Tyrosine Kinases and Protein Serine/Threonine Kinases, based, on the amino acid residue they phosphorylate. The serine/threonine kinases (PSTK), includes cyclic AMP- and cyclic GMP-dependent protein kinases, calcium- and phospholipid-dependent protein kinase, calcium- and calmodulin-dependent protein kinases, casein kinases, cell division cycle protein kinases and others. These kinases are usually cytoplasmic or associated with the particulate fractions of cells, possibly by anchoring proteins. Aberrant protein serine/threonine kinase activity has been implicated or is suspected in a number of pathologies such as rheumatoid arthritis, psoriasis, septic shock, bone loss, many cancers and other proliferative diseases. Accordingly, serine/threonine kinases and the signal transduction pathways which they are part of are important targets for drug design. The tyrosine kinases phosphorylate tyrosine residues. Tyrosine kinases play an equally important role in cell regulation. These kinases include several receptors for molecules such as growth factors and hormones, including epidermal growth factor receptor, insulin receptor, platelet derived growth factor receptor and others. Studies have indicated that many tyrosine kinases are transmembrane proteins with their receptor domains located on the outside of the cell and their kinase domains on the inside. Much work is also under progress to identify modulators of tyrosine kinases as well.

A major signal transduction systems utilized by cells is the RhoA-signalling pathways. RhoA is a small GTP binding protein that can be activated by several extracellular stimuli such as growth factor, hormones, mechanic stress, osmotic change as well as high concentration of metabolite like glucose. RhoA activation involves GTP binding, conformation alteration, post-translational modification (geranylgeranyllization and farnesylation) and activation of its intrinsic GTPase activity. Activated RhoA is capable of interacting with several effector proteins including Rho-Kinases (ROCK 1 and ROCK 2, referred to below as ‘ROCK’ or ‘ROCKs’) and transmit signals into cellular cytoplasm and nucleus.

ROCK1 and 2 constitute a family of kinases that can be activated by RhoA-GTP complex via physical association. Activated ROCKs phosphorylate a number of substrates and play important roles in pivotal cellular functions. The substrates for ROCKs include myosin binding subunit of myosin light chain phosphatase (MBS, also named MYPT1), adducin, moesin, myosin light chain (MLC), LIM kinase as well as transcription factor FHL. The phosphorylation of theses substrates modulate the biological activity of the proteins and thus provide a means to alter cell's response to external stimuli. One well documented example is the participation of ROCK in smooth muscle contraction. Upon stimulation by phenylephrine, smooth muscle from blood vessels contracts. Studies have shown that phenylephrine stimulates b-adrenergic receptors and leads to the activation of RhoA. Activated RhoA in turn stimulates kinase activity of ROCK1 and which in turn phosphorylates NBS. Such phosphorylation inhibits the enzyme activity of myosin light chain phosphatase and increases the phosphorylation of myosin light chain itself by a calcium-dependent myosin light chain kinase (MLCK) and consequently increases the contractility of myosin-actin bundle, leading to smooth muscle contraction. This phenomena is also sometimes called calcium sensitization. In addition to smooth muscle contraction, ROCKs have also been shown to be involved in cellular functions including apoptosis, cell migration, transcriptional activation, fibrosis, cytokinesis, inflammation and cell proliferation. Moreover, in neurons ROCK plays a critical role in the inhibition of axonal growth by myelin-associated inhibitory factors such as myelin-associated glycoprotein (MAG). ROCK-activity also mediates the collapse of growth cones in developing neurons. Both processes are thought to be mediated by ROCK-induced phosphorylation of substrates such as LIM kinase and myosin light chain phosphatase, resulting in increased contractility of the neuronal actin-myosin system.

The present inventors have discovered novel azabenzimidazole compounds, which are inhibitors of ROCK activity and show interesting selectivity over other protein kinases. Such derivatives are useful in the treatment of disorders associated with inappropriate ROCK activity.

### DETAILED DESCRIPTION OF THE INVENTION

The present invention includes compounds as described hereinbelow: The present invention thus provides compounds of the general formula (I) 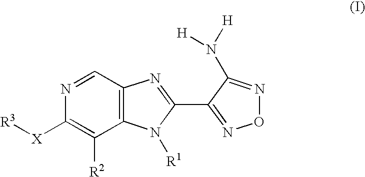
and physiologically acceptable salts wherein,
X represents O or R³X represents F, Cl, or Br;
R¹ represents hydrogen or C₁₋₆ alkyl;
R² represents Cl, Br, or I, optionally substituted phenyl, heteroaryl, or CONR⁴R⁵;
R³ represents C₁₋₆ alkyl, optionally substituted by a group selected from the group consisting of optionally substituted phenyl, C₃₋₇cycloalkyl, heteroaryl, heterocyclyl, NH₂, R⁴R⁵N, acylamino, hydroxy, CO₂R⁴, CONR⁴R⁵, NR⁴COR⁵, NR⁴CSR⁵, SO₂NR⁴R⁵, NR⁴SO₂R⁵, and OalkNR⁴R⁵ optionally substituted phenyl, heteroaryl, or heterocyclyl;
R⁴ and R⁵, independently represent a group selected from hydrogen, C₁₋₆ alkyl, C₃₋₇ cycloalkyl, C₃₋₇ cycloalkylalkyl, aryl, aralkyl, heteroaryl, heteroarylalkyl, heterocyclyl and heterocyclylalkyl;
alk is a C₂₋₄ straight or branched alkylene chain.

It will be appreciated that any of the substituents R¹ to R⁵ as defined in formula (I) above may contain at least one asymmetric center and it is to be understood that the invention includes all possible enantiomers arising therefrom and mixtures thereof including racemates.

The present invention thus provides compounds of the general formula (II) 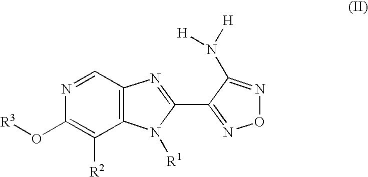
and physiologically acceptable salts wherein,
R¹ represents C₁₋₄ alkyl;
R² represents Br, optionally substituted phenyl or CONR⁴R⁵;
R³ represents optionally substituted phenyl or heteroaryl;
R⁴ and R⁵ independently represent a group selected from hydrogen, optionally substituted C₁₋₆ alkyl, optionally substituted C₃₋₇ cycloalkyl, optionally substituted C₃₋₇ cycloalkylalkyl, heterocyclyl and heterocyclylalkyl, or R⁴ and R⁵ together form a ring;

It will be appreciated that any of the substituents R¹ to R⁵ as defined in formula (I) above may contain at least one asymmetric center and it is to be understood that the invention includes all possible enantiomers arising therefrom and mixtures thereof including racemates.

The term alkyl as a group or part of a group e.g. alkoxy, alkylthio, alkylamino, dialkylamino, optionally substituted alkyl e.g. aminoalkyl, cycloalkylalkyl, aralkyl, heteroarylalkyl or heterocyclylalkyl refers to a C₁₋₆ straight or branched chain alkyl group.

The term halogen includes fluorine, chlorine, bromine or iodine.

The term aryl as a group or part of a group e.g. aryloxy, aralkyl or arylamino refers to an optionally substituted phenyl or fused bicyclic aryl group e.g. naphthyl.

The terms aryl, optionally substituted phenyl, heteroaryl, C₃₋₇ cycloalkyl as a group or part of a group and 4-7 membered heterocyclyl as a group or part of a group includes such groups which are optionally substituted with 1 to 3 substituents which may be the same or different and selected from halogen, aryl, heteroaryl, heterocyclylalkyl, hydroxy, alkyl, alkoxy, trifluoroalkyl, amino, alkylamino, dialkylamino, arylamino, heteroarylamino, heterocyclylamino, acylamino, aminoalkyl, alkylaminoalkyl, dialkylaminoalkyl, acylaminoalkyl, arylaminoalkyl, heteroarylaminoalkyl, cycloalkylaminoalkyl, heteroclylaminoalkyl, hydroxyalkyl, CONR₄R₅, CSNR₄R₅, CH₂CONR₄R₅, carboxy, carboxamido, alkoxycarbonyl, aminoalkoxy, dialkylaminoalkoxy, acylaminoalkoxy, sulphonamido, aminosulphonyl, cyano, formyl, nitro, R⁶⁰ or R⁶S(O)ₙ wherein R⁶ is a group selected from alkyl, aryl, heteroaryl or heterocyclylalkoxy and n is zero, one or two, or each of the said groups can form part of a fused bicyclic ring system containing up to 10 ring members and which can be at least partially saturated.

The term heteroaryl as a group or part of a group e.g. heteroaryloxy refers to a 5, or 6 membered ring or a fused 5,6 or 6,6 bicyclic ring system.

When heteroaryl represents a 5 membered group it contains a heteroatom selected from O, N or S and may optionally contain a further 1 to 3 nitrogen atoms. Examples of such groups include furanyl, thienyl, isoxazolyl, oxazolyl or imidazolyl.

When heteroaryl represents a 6-membered group it contains from 1 to 3 nitrogen atoms. Examples of such groups include pyridyl, pyrimidinyl, or triazinyl. The term 5,6 fused bicyclic heteroaryl group refers to a group in which the 5-membered ring contains an oxygen, sulphur or NH group and may optionally contain a further 1 to 2 nitrogen atoms, and the 6 membered ring optionally contains from 1 to 3 nitrogen atoms. Examples of such groups include benzofuranyl, benzothienyl, benzimidazole, benzotriazole or indolyl.

The term 6,6-fused bicyclic heteroaryl group refers to a bicyclic heteroaryl group which contains at least one nitrogen atom in one of the rings and may contain up to 3 nitrogen atoms in each ring. Examples of such groups include quinolinyl, isoquinolinyl or naphthyridinyl also the term 6,6 fused bicyclic heteroaryl group refers to a 6-membered heteroaryl group which is fused to a partially saturated carbocyclic group. Examples of such a group includes tetahydroquinolinyl or tetrahydroisoquinolinyl.

The term heterocyclyl as a group or part of a group e.g. heterocyclylalkyl or heterocyclylalkylidene refers to a bridged heterocyclic group or a 4-7 membered heterocyclyl group which is linked to the rest of the compound of formula (1) via a carbon or nitrogen atom in that group and which contains one or two hetero atoms selected from N, O or S(O)ₙ, and when the heterocyclyl group contains a ring member NH or the heterocyclyl group is substituted by a primary or secondary amino group then the term also includes N-alkyl, N-optionally substituted phenyl, N-araalkyl, N-sulfonyl, or, N-acyl derivatives thereof. The term heterocyclic also includes bridged heterocyclic. Examples of such heterocyclic groups include optionally substituted pyrrolidine, piperidine, piperazine, homopiperazine, morpholine, thiomorpholine and (8-methyl-8-aza-bicyclo[3.2.1]oct-3-yl)-amine.

The term cycloalkyl as a group or part of a group e.g. cycloalkylalkyl or cycloalkylidene refers to a 3-7 membered carbocyclic group. The term fused bicyclic ring system containing up to 11 ring members and which is at least partially saturated includes carbocyclic and heterocyclic 6,5, 6,6 and 6,7 bicyclic ring systems. Examples of such 6,5 and 6,6 carbocyclic ring systems include those wherein the bicyclic ring comprises a benzene ring fused to a 5-, 6- or -membered carbocyclic ring which is at least partially saturated e.g. tetrahydronaphthyl, indanyl or indenyl. Examples of such 6,5, 6,6 or 6,7 heterocyclic rings include those wherein one ring is benzene which is fused to a 5, 6 or 7 membered ring containing one or two hetero atoms selected from O, S or N e.g. indolinyl, isoindolinyl, 2,3-dihydro-1H-isoindol-5-yl, dihydrobenzofuranyl, dihydrobenzothienyl, 1,3-benzodioxolyl, benzopyrrolyl, 1,3-benozodithiolyl, 1,4-benzodioxanyl, chromanyl, chromenyl or 2,3,4,5-tetrahydro-1H-benzo[c]azepin-8-yl.

The term acyl as a group or part of the acylamino group refers to an alkanoyl, aroyl, aralkanoyl, alkoxycarbonyl, aryloxycaronyl or aralkoxycarbonyl group.

The compounds of formula (I) form salts with inorganic and organic acids and the invention includes such salts formed with physiologically acceptable inorganic and organic acids.

A preferred example of R¹ includes, but is not limited to, C₁₋₆alkyl, e.g. ethyl. Preferred examples of R² include, but are not limited to, optionally substituted phenyl (e.g. phenyl or phenyl substituted by one or two groups selected from halogen e.g., fluoro or alkylaminoalkyl, e.g. ethylaminomethyl), heterocyclic amides, e.g. 3-aminopyrrolidin-1-yl-carbonyl.

A preferred example of R³ includes, but is not limited to, optionally substituted phenyl (e.g. 4-fluorophenyl).

A preferred example of X is O.

Inhibitors of ROCKs have been suggested for use in the treatments of a variety of diseases. They include cardiovascular diseases such as hypertension, chronic and congestive heart failure, ischemic angina, cardiac hypertrophy and fibrosis, restenosis, chronic renal failure and atherosclerosis. In addition, because of its muscle relaxing properties, it is also suitable for asthma, male erectile dysfunctions, female sexual dysfunction and over-active bladder syndrome. ROCK inhibitors have been shown to possess anti-inflammatory properties. Thus they can be used as treatment for neuroinflammatory diseases such as stroke, multiple sclerosis, Alzheimer's disease, Parkinson's disease, amyotrophic lateral sclerosis and inflammatory pain, as well as other inflammatory diseases such as rheumatoid arthritis, irritable bowel syndrome, inflammatory bowel disease, and Crohn's diseases. In addition, based on their neurite outgrowth inducing effects, ROCK inhibitors could be useful drugs for neuronal regeneration, inducing new axonal growth and axonal rewiring across lesions within the CNS. ROCK inhibitors are therefore likely to be useful for regenerative (recovery) treatment of CNS disorders such as spinal cord injury, acute neuronal injury (stroke, traumatic brain injury), Parkinsons disease, Alzheimers disease and other neurodegenerative disorders. Since ROCK inhibitors reduce cell proliferation and cell migration, they could be useful in treating cancer and tumor metastasis. Further more, there is evidence suggesting that ROCK inhibitors suppress cytoskeletal rearrangement upon virus invasion, thus they also have potential therapeutic value in anti-viral and anti-bacterial applications. ROCK inhibitors are also useful for the treatment of insulin resistance and diabetes.

Preferably ROCK inhibitors are useful for the treatment of hypertension, chronic and congestive heart failure, ischemic angina, asthma, male erectile dysfunction, female sexual dysfunction, stroke, inflammatory bowel diseases, spinal cord injury, glaucoma and tumor metastasis.

More preferably ROCK inhibitors are useful for the treatment of hypertension, chronic and congestive heart failure and ischemic angina.

As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

As used herein, the term “optionally” means that the subsequently described event(s) may or may not occur, and includes both event(s), which occur, and events that do not occur.

As used herein, the term “physiologically functional derivative” refers to any pharmaceutically acceptable derivative of a compound of the present invention, for example, an ester or an amide, which upon administration to a mammal is capable of providing (directly or indirectly) a compound of the present invention or an active metabolite thereof. Such derivatives are clear to those skilled in the art, without undue experimentation, and with reference to the teaching of Burger's Medicinal Chemistry And Drug Discovery, 5^{th} Edition, Vol 1: Principles and Practice, which is incorporated herein by reference to the extent that it teaches physiologically functional derivatives.

As used herein, the term “solvate” refers to a complex of variable stoichiometry formed by a solute (in this invention, a compound of formula (I) or a salt or physiologically functional derivative thereof) and a solvent Such solvents for the purpose of the invention may not interfere with the biological activity of the solute. Examples of suitable solvents include, but are not limited to, water, methanol, ethanol and acetic acid. Preferably the solvent used is a pharmaceutically acceptable solvent. Examples of suitable pharmaceutically acceptable solvents include, without limitation, water, ethanol and acetic acid. Most preferably the solvent used is water.

As used herein, the term “substituted” refers to substitution with the named substituent or substituents, multiple degrees of substitution being allowed unless otherwise stated.

Certain of the compounds described herein may contain one or more chiral atoms, or may otherwise be capable of existing as two enantiomers. The compounds of this invention include mixtures of enantiomers as well as purified enantiomers or enantiomerically enriched mixtures. Also included within the scope of the invention are the individual isomers of the compounds represented by formula (I) above as well as any wholly or partially equilibrated mixtures thereof. The present invention also covers the individual isomers of the compounds represented by the formulas above as mixtures with isomers thereof in which one or more chiral centers are inverted. Also, it is understood that any tautomers and mixtures of tautomers of the compounds of formula (I) are included within the scope of the compounds of formula (I).

Typically, the salts of the present invention are pharmaceutically acceptable salts. Salts encompassed within the term “pharmaceutically acceptable salts” refer to non-toxic salts of the compounds of this invention. Salts of the compounds of the present invention may comprise acid addition salts derived from a nitrogen on a substituent in the compound of formula (I). Representative salts include the following salts: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, laurate, malate, maleate, mandelate, mesylate, methylbromide, methylnitrate, methylsulfate, monopotassium maleate, mucate, napsylate, nitrate, N-methylglucamine, oxalate, pamoate (embonate), palmitate, pantothenate, phosphate/diphosphate, polygalacturonate, potassium, salicylate, sodium, stearate, subacetate, succinate, tannate, tartrate, teoclate, tosylate, triethiodide, trimethylammonium and valerate. Other salts, which are not pharmaceutically acceptable, may be useful in the preparation of compounds of this invention and these form a further aspect of the invention.

While it is possible that, for use in therapy, therapeutically effective amounts of a compound of formula (I), as well as salts, solvates and physiological functional derivatives thereof, may be administered as the raw chemical, it is possible to present the active ingredient as a pharmaceutical composition. Accordingly, the invention further provides pharmaceutical compositions, which include therapeutically effective amounts of compounds of the formula (I) and salts, solvates and physiological functional derivatives thereof, and one or more pharmaceutically acceptable carriers, diluents, or excipients. The compounds of the formula (I) and salts, solvates and physiological functional derivatives thereof, are as described above. The carrier(s), diluent(s) or excipient(s) must be acceptable in the sense of being compatible with the other ingredients of the formulation and not deleterious to the recipient thereof. In accordance with another aspect of the invention there is also provided a process for the preparation of a pharmaceutical formulation including admixing a compound of the formula (I), or salts, solvates and physiological functional derivatives thereof, with one or more pharmaceutically acceptable carriers, diluents or excipients.

Pharmaceutical formulations may be presented in unit dose forms containing a predetermined amount of active ingredient per unit dose. Such a unit may contain, for example, 0.5 mg to 1 g, preferably 1 mg to 700 mg, more preferably 5 mg to 100 mg of a compound of the formula (I), depending on the condition being treated, the route of administration and the age, weight and condition of the patient, or pharmaceutical formulations may be presented in unit dose forms containing a predetermined amount of active ingredient per unit dose. Preferred unit dosage formulations are those containing a daily dose or sub-dose, as herein above recited, or an appropriate fraction thereof, of an active ingredient. Furthermore, such pharmaceutical formulations may be prepared by any of the methods well known in the pharmacy art.

Pharmaceutical formulations may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such formulations may be prepared by any method known in the art of pharmacy, for example by bringing into association the active ingredient with the carrier(s) or excipient(s).

Pharmaceutical formulations adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

For instance, for oral administration in the form of a tablet or capsule, the active drug component can be combined with an oral, non-toxic pharmaceutically acceptable inert carrier such as ethanol, glycerol, water and the like. Powders are prepared by comminuting the compound to a suitable fine size and mixing with a similarly comminuted pharmaceutical carrier such as an edible carbohydrate, as, for example, starch or mannitol. Flavoring, preservative, dispersing and coloring agent can also be present.

Capsules are made by preparing a powder mixture, as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an aliginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by wetting with a binder such as syrup, starch paste, acadia mucilage or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solution, syrups and elixirs can be prepared in dosage unit form so that a given quantity contains a predetermined amount of the compound. Syrups can be prepared by dissolving the compound in a suitably flavored aqueous solution, while elixirs are prepared through the use of a non-toxic alcoholic vehicle. Suspensions can be formulated by dispersing the compound in a non-toxic vehicle. Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

Where appropriate, dosage unit formulations for oral administration can be microencapsulated. The formulation can also be prepared to prolong or sustain the release as for example by coating or embedding particulate material in polymers, wax or the like.

The compounds of formula (I), and salts, solvates and physiological functional derivatives thereof, can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The compounds of formula (I) and salts, solvates and physiological functional derivatives thereof may also be delivered by the use of monoclonal antibodies as individual carriers to which the compound molecules are coupled. The compounds may also be coupled with:soluble polymers as targetable drug carriers. Such polymers can include polyvinylpyrrolidone, pyran copolymer, polyhydroxypropylmethacrylamide-phenol, polyhydroxyethylaspammidephenol, or polyethyleneoxidepolylysine substituted with palmitoyl residues. Furthermore, the compounds may be coupled to a class of biodegradable polymers useful in achieving controlled release of a drug, for example, polylactic acid, polepsilon caprolactone, polyhydroxy butyric acid, polyorthoesters, polyacetals, polydihydropyrans, polycyanoacrylates and cross-linked or amphipathic block copolymers of hydrogels.

Pharmaceutical formulations adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research, 3(6), 318 (1986).

Pharmaceutical formulations adapted for topical administration may be formulated as ointments, creams, suspensions, lotions, powders, solutions, pastes, gels, sprays, aerosols or oils.

For treatments of the eye or other external tissues, for example mouth and skin, the formulations are preferably applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base.

Pharmaceutical formulations adapted for topical administrations to the eye include eye drops wherein the active ingredient is dissolved or suspended in a suitable carrier, especially an aqueous solvent.

Pharmaceutical formulations adapted for topical administration in the mouth include lozenges, pastilles and mouth washes.

Pharmaceutical formulations adapted for rectal administration may be presented as suppositories or as enemas.

Pharmaceutical formulations adapted for nasal administration wherein the carrier is a solid include a coarse powder having a particle size for example in the range 20 to 500 microns which is administered in the manner in which snuff is taken, i.e. by rapid inhalation through the nasal passage from a container of the powder held close up to the nose. Suitable formulations wherein the carrier is a liquid, for administration as a nasal spray or as nasal drops, include aqueous or oil solutions of the active ingredient.

Pharmaceutical formulations adapted for administration by inhalation include fine particle dusts or mists, which may be generated by means of various types of metered, dose pressurised aerosols, nebulizers or insufflators.

Pharmaceutical formulations adapted for vaginal administration may be presented as pessaries, tampons, creams, gels, pastes, foams or spray formulations.

Pharmaceutical formulations adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

It should be understood that in addition to the ingredients particularly mentioned above, the formulations may include other agents conventional in the art having regard to the type of formulation in question, for example those suitable for oral administration may include flavouring agents.

A therapeutically effective amount of a compound of the present invention will depend upon a number of factors including, for example, the age and weight of the human or other animal, the precise condition requiring treatment and its severity, the nature of the formulation, and the route of administration, and will ultimately be at the discretion of the attendant physician or veterinarian. However, an effective amount of a compound of formula (I) for the treatment of neoplastic growth, for example colon or breast carcinoma, will generally be in the range of 0.1 to 100 mg/kg body weight of recipient (mammal) per day and more usually in the range of 1 to 10 mg/kg body weight per day. Thus, for a 70 kg adult mammal, the actual amount per day would usually be from 70 to 700 mg and this amount may be given in a single dose per day or more usually in a number (such as two, three, four, five or six) of sub-doses per day such that the total daily dose is the same. An effective amount of a salt or solvate, or physiologically functional derivative thereof, may be determined as a proportion of the effective amount of the compound of formula (I) per se. It is envisaged that similar dosages would be appropriate for treatment of the other conditions referred to above.

The compounds of this invention may be made by a variety of methods, including standard chemistry. Any previously defined variable will continue to have the previously defined meaning unless otherwise indicated. Illustrative general synthetic methods are set out below and then specific compounds of the invention are prepared in the Working Examples.

Compounds of general formula (I) may be prepared by methods known in the art of organic synthesis as set forth in part by the following synthesis schemes. In all of the schemes described below, it is well understood that protecting groups for sensitive or reactive groups are employed where necessary in accordance with general principles of chemistry. Protecting groups are manipulated according to standard methods of organic synthesis (T. W. Green and P. G. M. Wuts (1991) Protecting Groups in Organic Synthesis, John Wiley & Sons). These groups are removed at a convenient stage of the compound synthesis using methods that are readily apparent to those skilled in the art. The selection of processes as well as the reaction conditions and order of their execution shall be consistent with the preparation of compounds of Formula (I).

Compounds with the general structure 8 can be prepared according to the procedure described in Scheme 1. Treatment of an appropriately substituted pyridine derivative 1 with potassium t-butoxide and t-butyl hydroperoxide provides the pyridone 2 which can be chlorinated and the resulting dichloride treated with a primary amine to give the aminopyridine 3. Displacement of the second chloride with a sodium salt, followed by reduction provides structure 5, which can then be coupled to cyanoacetic acid with a variety of coupling agents to provide the corresponding cyanoacetamide (not shown). This amide can then be dehydrated with glacial acetic acid to give the azabenzimidazole 6. The nitrile 6 can be transformed into the oxime 7 by treatment with nitrous acid and then further elaborated to the aminofurazan structure 8 by treatment with hydroxylamine and base.

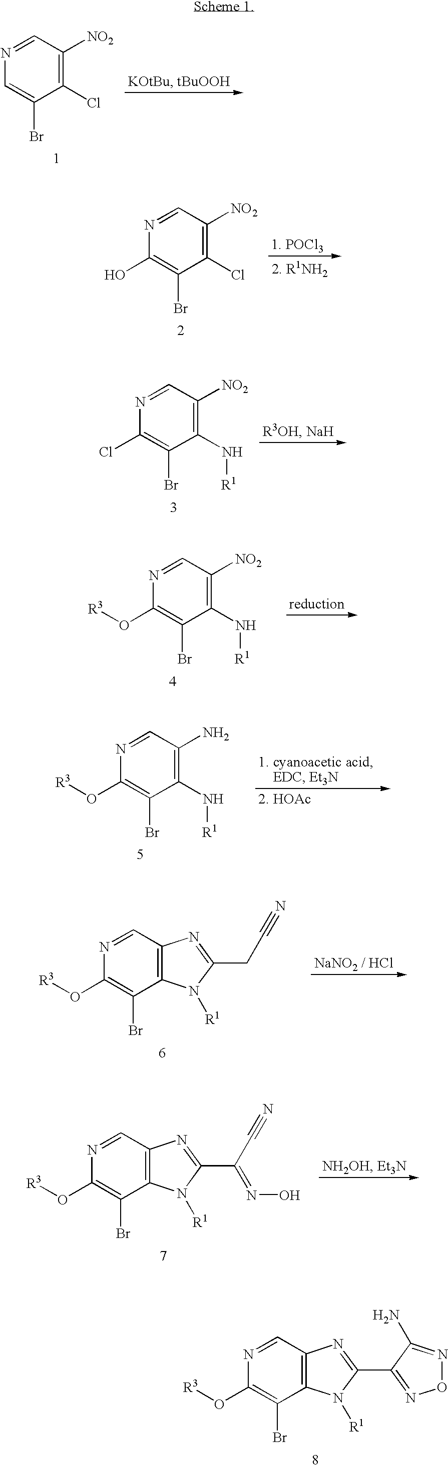

Compounds of the general structure 8 can be further transformed to a variety of structures, Scheme 2. Palladium-catalyzed coupling can provide compounds such as 9. Alternatively, treatment with LDA followed by BuLi provides the corresponding lithium reagent, which can be quenched with a variety of electrophiles, for example carbon dioxide to give an acid which can then be coupling with a variety of amines to give the amides 10.

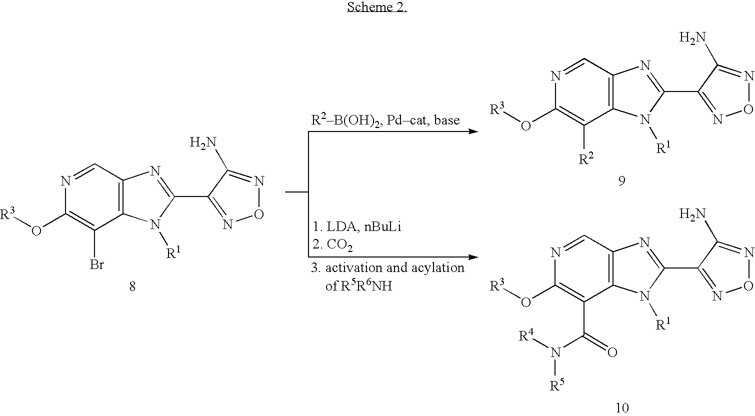

Examples of suitable compounds according to the invention include those listed below and found in Examples 1-4. These are intended to be illustrative only and not limiting in any way.
4-{1-Ethyl-6-[(4-fluorophenyl)oxy]-7-phenyl-1H-imidazo[4,5-c]pyridin-2-yl}-furazan-3-amine,
4-{1-Ethyl-7-(4-fluorophenyl)-6-[(4-fluorophenyl)oxy]-1H-imidazo[4,5-c]pyridin-2-yl}-furazan-3-amine,
4-{1-Ethyl-7-{3-[(ethylamino)methyl]phenyl}-6-[(4-fluorophenyl)oxy]-1H-imidazo[4,5-c]pyridin-2-yl}-furazan-3-amine,
4-{7-{[(3S)-3-Amino-1-pyrrolidinyl]carbonyl}-1-ethyl-6-[(4-fluorophenyl)oxy]-1H-imidazo[4,5-c]pyridin-2-yl}-furazan-3-amine;
1,1-dimethylethyl(3-{[2-(4-amino-furazan-3-yl)-7-bromo-1-ethyl-1H-imidazo[4,5-c]pyridin-6-yl]oxy}phenyl)carbamate;
N-(3-{[2-(4-amino-furazan-3-yl)-7-bromo-1-ethyl-1H-imidazo[4,5-c]pyridin-6-yl]oxy}phenyl)acetamide

### EXAMPLES

The following examples are intended to be illustrative only and not limiting in any way:

### Example 1

### 4-{1-Ethyl-6-[(4-fluorophenyl)oxy]-7-phenyl-1H-imidazo[4,5-c]pyridin-2-yl}-furazan-3-amine

### Step 1. 3-Bromo-5-nitro-4-pyridone

3-Nitro-4-pyridone (50.0 g, 357 mmol) was suspended in H₂O (500 mL) and bromine (23.0 mL, 446 mmol) was added dropwise, then the heterogeneous mixture was warmed to 70° C. for 2 h, then cooled to 0° C. and filtered. The resulting solid was washed with H₂O and dried to give the title compound as a tan solid (62.0 g, 79%).

### Step 2. 3-Bromo-4-chloro-5-nitropyridine

The product from Step 1 (25.0 g, 114 mmol) was suspended in toluene (50 mL) and POCl₃ (53 mL, 570 mmol) was added and the slurry was heated to 90° C. overnight. The mixture was cooled and concentrated and the residue was carefully quenched with ice and sat. K₂CO₃ solution then extracted with EtOAc. The organic layers were combined, washed with H₂O and brine, dried (MgSO₄), filtered and concentrated to give the title compound (26.1 g, 97%) as tan oil which crystallized on standing. MS (ES+) m/e 237, 239 [M+H]⁺.

### Step 3. 3-Bromo-4-chloro-5-nitro-2-pyridone

THF (500 mL) was cooled to −78° C. and anhydrous NH₃ (˜200 mL) was condensed into the THF. Potassium t-butoxide (31.0 g, 275 mmol) was added and the mixture was allowed to warm to ˜−35° C. The product from Step 2 (26.0 g, 110 mmol) was cooled to 0° C. in THF (200 mL) and a solution of t-BuOOH (5 M in decane, 22 mL, 110 mmol) was added over 5 min. This solution was then added dropwise to the KOt-Bu solution over 1 h, then stirred for 1 h at −35° C. and then carefully quenched with ˜50 mL of sat. NH₄Cl solution. The mixture was allowed to vent and warm to rt overnight, then the organics were concentrated and the residue made acidic with NH₄Cl solution and filtered. The solid was washed with cold H₂O and dried to give the title compound as a dark brown solid (26.8 g, 96%).

### Step 4. 3-Bromo-2,4-dichloro-5-nitropyridine

The product from Step 3 (26.8 g, 106 mmol) was suspended in acetonitrile 300 m), POCl₃ (40 mL, 418 mmol) was added and the mixture was heated to 90° C. overnight. The mixture was cooled and concentrated and the residue was carefully quenched with ice and sat. K₂CO₃ solution then extracted with EtOAc. The organic layers were combined, washed with H₂O and brine, dried (MgSO₄), filtered and concentrated to give the title compound (18 g, 66%) as a dark oil. MS (ES+) m/e 272, 274 [M+H]⁺.

### Step 5. 3-Bromo-2-chloro-4-ethylamino-5-nitropyridine

The product from Step 4 (18 g, 66 mmol) was dissolved in THF (150 mL) and Et₃N (9.8 mL, 70 mmol) was added, followed by ethylamine (2M in THF, 33 mL, 66 mmol) and the mixture was stirred at rt for 2 h, then poured into H₂O and extracted with EtOAc. The organic layers were combined, washed with H₂O and brine, dried (MgSO₄), filtered and concentrated to give the title compound (16.5 g, 89%) as an orange oil. MS (ES+) m/e 280, 282 [M+H]⁺.

### Step 6. 3-Bromo-4-ethylamino-2-(4-fluorophenoxy)-5-nitropyridine

The product of Step 5 (10.0 g, 35.6 mmol), 4-fluorophenol (4.4 g, 39.2 mmol) and K₂CO₃ (10 g) were combined in acetonitrile (100 mL) and heated to 80° C. over the weekend. The mixture was then poured into H₂O and extracted with EtOAc. The organic layers were combined, washed with H₂O and brine, dried (MgSO₄), filtered and concentrated to give an oil which was chromatographed (5-30% EtOAc in hex) to give the title compound (10.4 g, 82%) as yellow solid. MS (ES+) m/e 356, 358 [M+H]⁺.

### Step 7. 5-Bromo-M-ethyl-6-[(4-fluorophenyl)oxy]-3,4-pyridinediamine

The product from Step 6 (2.7 g, 7.6 mmol) was dissolved in EtOH (30 mL) and 1 mL conc. HCl was added, followed by SnCl₂ (10.0 g) portionwise. The mixture was stirred at rt for 2 h, then concentrated and the residue quenched with sat. K₂CO₃ solution then extracted with EtOAc. The organic layers were combined, washed with H₂O and brine, dried (MgSO₄), filtered and concentrated to give the title compound (2.3 g, 91%) as dark oil. MS (ES+) m/e 326, 328 [M+H]⁺.

### Step 8. N-{5-Bromo-4-(ethylamino)-6-[(4-fluorophenyl)oxy]-3-pyridinyl}-2-cyanoacetamide

The product from Step 7 (2.24 g, 6.9 mmol), cyanoacetic acid (1.17 g, 13.7 mmol) and EDC (2.60 g, 13.7 mmol) were combined in CH₂Cl₂ (25 mL) and Et₃N (3.8 mL, 27.4 mmol) was added and the mixture was allowed to stir at rt overnight. The mixture was then poured into H₂O, extracted with EtOAc, the organic layers were combined, washed with H₂O and brine, dried (MgSO₄), filtered and concentrated to give the title compound (2.7 g, 99%) as dark solid which was used without purification. MS (ES+) m/e 393, 395 [M+H]⁺.

### Step 9. {7-Bromo-1-ethyl-6-[(4-fluorophenyl)oxy]-1H-imidazo[4,5-c]pyridin-2-yl}acetonitrile

The product from Step 8 (2.7 g, 6.9 mmol) was heated to 100° C. in HOAc for 1 h, then cooled and concentrated. The residue was made basic with ice and sat. K₂CO₃ solution then extracted with EtOAc. The organic layers were combined, washed with H₂O and brine, dried (MgSO₄), filtered and concentrated and the residue purified by chromatography to give the title compound (2.25 g, 86%) as a tan solid. MS (ES+) m/e 375, 377 [M+H]⁺.

### Step 10. 4-{7-Bromo-1-ethyl-6-[(4-fluorophenyl)oxy]-1H-imidazo[4,5-c]pyridin-2-yl}-furazan-3-amine

The product of Step 9 (2.25 g, 6 mmol) was dissolved in MeOH (20 mL) and 6M HCl (3 mL) was added, followed by NaNO₂ (0.414 g, 6 mmol, in ˜100 mg portions). The mixture was stirred for 1 h and the resulting precipitate was filtered and dried then suspended in dioxane (15 mL) and combined with NH₂OH (50% aq. solution, 600 uL) and Et₃N (3 mL) and heated to 100° C. for 1 h. The solution was then cooled, poured into H₂O and extracted with EtOAc. The organic layers were combined, washed with brine, dried (MgSO₄), filtered and concentrated to give a solid which was again suspended in dioxane (10 mL) and combined with Et₃N (3 mL) then heated to 140° C. in a sealed tube for 3 h, then cooled and poured into H₂O. The mixture was extracted with EtOAc, the organic layers were combined, washed with brine, dried (MgSO₄), filtered and concentrated to give the title compound (1.3 g, 52%) as an off white solid. MS (ES+) m/e 419, 421 [M+H]⁺.

### Step 11. 4-{1-Ethyl-6-[(4-fluorophenyl)oxy]-7-phenyl-1H-imidazo[4,5-c]pyridin-2-yl}-furazan-3-amine

The product from Step 10 (100 mg, 0.24 mmol) was combined with phenylboronic acid (44 mg, 0.35 mmol) and (dppf)₂PdCl₂ (20 mg) in dioxane (2 mL) and 2M Na₂CO₃ (0.3 mL) and heated to 90° C. overnight The mixture was cooled, poured into H₂O and extracted with EtOAc, the organic layers were combined, washed with brine, dried (MgSO₄), filtered, concentrated and the crude material was purified by reverse-phase HPLC to give the title compound (45 mg, 45%) as a tan solid. MS (ES+) m/e 417 [M+H]⁺.

### Example 2

### 4-{1-Ethyl-7-(4-fluorophenyl)-6-[(4-fluorophenyl)oxy]-1H-imidazo[4,5-c]pyridin-2-yl}-furazan-3-amine

The product from Example 1, Step 10 was treated as in Example 1, Step 11, except using 4-fluorophenylboronic acid to give the title compound as a tan solid. MS (ES+) m/e 435 [M+]⁺.

### Example 3

### 4-{1-Ethyl-7-{3-[(ethylamino)methyl]phenyl}-6-[(4-fluorophenyl)oxy]-1H-imidazo[4,5-c]pyridin-2-yl}-furazan-3-amine

### Step 1. 3-{2-(4-amino-furazan-3-yl)-1-ethyl-6-[(4-fluorophenyl)oxy]-1H-imidazo[4,5-c]pyridin-7-yl}benzaldehyde

The product from Example 1, Step 10 was treated as in Example 1, Step 11, except using 3-formylbenzeneboronic acid to give the title compound as a brown solid. MS (ES+) m/e 445 [M+H]⁺.

### Step 2. 4-{1-Ethyl-7-{3-[(ethylamino)methyl]phenyl}-6-[(4-fluorophenyl)oxy]-1H-imidazo[4,5-c]pyridin-2-yl}-furazan-3-amine

The product from Step 1 (106 mg, 0.24 mmol) was dissolved in MeOH (2 mL), treated with EtNH₂ (70% aq. 100 uL) and Na(OAc)₃BH and allowed to stir at rt overnight. The mixture was concentrated and the residue purified by reverse-phase HPLC to give the title compound (77 mg, 67%) as an off-white solid. MS (ES+) m/e 474 [M+H]⁺.

### Example 4

### 4-{7-([(3S)-3-Amino-1-pyrrolidinyl]carbonyl)-1-ethyl-6-[(4-fluorophenyl)oxy]-1H-imidazo[4,5-c]pyridin-2-yl}-furazan-3-amine

### Step 1. 2-(4-amino-furazan-3-yl)-1-ethyl-6-[(4-fluorophenyl)oxy]-1H-imidazo[4,5-c]pyridine-7-carboxylic acid

n-Butyllithium (2.5 M, 1.5 mL, 3.8 mmol) was added to a solution of i-Pr₂NH (560 uL, 4.0 mmol) in THF (15 mL). This solution was then added dropwise to a cold (−78° C.) solution of the product from Example 1, Step 10 (800 mg, 1.9 mmol) in THF (20 mL). The resulting mixture was stirred for 5 min, then n-butyllithium (2.5 M, 2:4 mL, 3.1 mmol) was added and the dark solution stirred an additional 10 min, then CO₂ gas was bubbled into the solution (quickly forming a yellow precipitate) and the mixture warmed to rt. The heterogeneous mixture was concentrated and dissolved in MeOH, then treated with aq. HCl and re-concentrated to give the title compound as a tan solid. MS (ES+) m/e 385 [M+H]⁺.

### Step 2. 4-{7-{[(3S)-3-Amino-1-pyrrolidinyl]carbonyl}-1-ethyl-6-[(4-fluorophenyl)oxy]-1H-imidazo[4,5-c]pyridin-2-yl}-furazan-3-amine

The product from Step 1 (200 mg, ˜0.5 mmol) was dissolved in DMF (2 mL) and treated with carbonyl diimidazole (240 mg, 1.5 mmol) and allowed to stir overnight at rt. The solution was then treated with 3-(S)—BOC-aminopyrrolidine (280 mg, 1.5 mmol) and allowed to stir overnight. The solution was poured into H₂O and extracted with EtOAc, the combined extracts were washed with brine, dried (MgSO₄), filtered, and concentrated to give an oil which was dissolved in CH₂Cl₂ (5 mL) and treated with TFA (1 mL) for 1 h, then concentrated. The crude product was purified by reverse-phase HPLC to give the title compound (89 mg, 39%) as a white solid. MS (ES+) m/e 453 [M+H]⁺.

### Example 5

### 1,1-dimethylethyl(3-{[2-(4-amino-furazan-3-yl)-7-bromo-1-ethyl-1H-imidazo[4,5-c]pyridin-6-yl]oxy}phenyl)carbamate

The title compound was prepared by the general method of Example 1, Steps 6-10, starting from 3-bromo-2-chloro-4-ethylamino-5-nitropyridine and 1,1-dimethylethyl(3-hydroxyphenyl)carbamate. MS (ES+) m/e 516/518 [M+H]⁺.

### Example 6

### N-(3-{[2-(4-amino-furazan-3-yl)-7-bromo-1-ethyl-1H-imidazo[4,5-c]pyridin-6-yl]oxy}phenyl)acetamide

The title compound was prepared by the general method of Example 1, Steps 6-10, starting from 3-bromo-2-chloro-4-ethylamino-5-nitropyridine and 3-acetamidophenol. MS (ES+) m/e 458/460 [M+H]⁺.

ROCK Kinase Assay:

ROCK inhibitor activity was determined using human recombinant ROCK1 kinase domain (amino acid 2-543) expressed in Sf9 cells (see WO9967283). The enzyme was purified using His-tag NTA column and Source 15 HPLC chromatography. The assay of Rock-1 activity involved incubation with peptide substrate and ATP³³, the subsequent incorporation of P³³ into the peptide was quantified by Scintillation Proximity Assay (SPA—Amersham Pharmacia).

For IC50 determination, test compounds were typically dissolved at 10 mM in 100% DMSO, with subsequent serial dilution in 100% DMSO. Compounds were typically assayed over an eleven-point dilution range with a concentration in the assay of 50 uM to 0.8 nM, in 3-fold dilutions. IC50 values were calculated by bespoke curve fitting software and then converted to pIC50.

Assays were performed in opaque, white walled, 384 well plates, in a total assay volume of 20 ul. The assays contained: 1 nM hROCK1; 1 uM biotinylated peptide (biotin-Ahx-AKRRRLSSLRA-CONH2); 1 uM ATP; 1.85 kBq per well ATP(□-33P); 25 mM Hepes pH 7.4; 15 mM MgCl₂; 0.015% BSA. The reactions were incubated at 22° C. for 120 minutes, then terminated by the addition of a 50 ul solution containing 60 mM EDTA and streptavidin PVT SPA beads. The SPA beads were added to a concentration of 0.14 mg per well. The plates were allowed to incubate at 22° C. for 10 minutes before centrifugation at 1500 rpm for 1 minute. P³³ incorporation was quantified by scintillation counting in a Packard TopCount.

All publications, including but not limited to patents and patent applications, cited in this specification are herein incorporated by reference as if each individual publication were specifically and individually indicated to be incorporated by reference herein as though fully set forth.

The above description fully discloses the invention including preferred embodiments thereof. Modifications and improvements of the embodiments specifically disclosed herein are within the scope of the following claims. Without further elaboration, it is believed that one skilled in the art can, using the preceding description, utilize the present invention to its fullest extent Therefore the Examples herein are to be construed as merely illustrative and not a limitation of the scope of the present invention in any way. The embodiments of the invention in which an exclusive property or privilege is claimed are defined as follows.

What is claimed is:

1. A compound according to formula (I) hereinbelow:
The present invention thus provides compounds of the general formula (I) 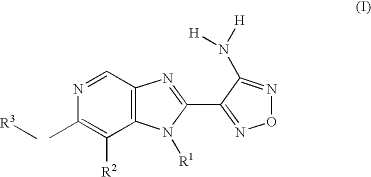
and physiologically acceptable salts wherein,
X represents O or R³X represents F, Cl, or Br;
R¹ represents hydrogen or C₁₋₆ alkyl;
R² represents Cl, Br, or I, optionally substituted phenyl, heteroaryl, or CONR⁴R⁵;
R³ represents C₁₋₆ alkyl, optionally substituted by a group selected from the group consisting of optionally substituted phenyl, C₃₋₇cycloalkyl, heteroaryl, heterocyclyl, NH₂, R⁴R⁵N, acylamino, hydroxy, CO₂R⁴, CONR⁴R⁵, NR⁴COR⁵ NR⁴CSR⁵, SO₂NR⁴R⁵, NR⁴SO₂R⁵, and OalkNR⁴R⁵ optionally substituted phenyl, heteroaryl, or heterocyclyl;
R⁴ and R⁵, independently represent a group selected from hydrogen, C₁₋₆ alkyl, C₃₋₇ cycloalkyl, C₃₋₇ cycloalkylalkyl, aryl, aralkyl, heteroaryl, heteroarylalkyl, heterocyclyl and heterocyclylalkyl;
alk is a C₂₋₄ straight or branched alkylene chain.

2. A compound according to claim 1 having general formula (II) 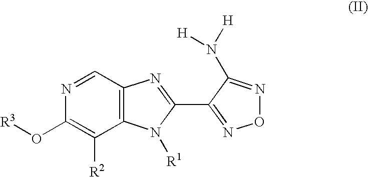
and physiologically acceptable salts wherein,
R¹ represents C₁₋₄ alkyl;
R² represents optionally substituted phenyl or CONR⁴R⁵;
R³ represents optionally substituted phenyl or heteroaryl;
R⁴ and R⁵ independently represent a group selected from hydrogen, optionally substituted C₁₋₆ alkyl, optionally substituted C₃₋₇ cycloalkyl, optionally substituted C₃₋₇ cycloalkylalkyl, heterocyclyl and heterocyclylalkyl, or R⁴ and R⁵ together form a ring.

3. A compound according to claim 1 selected from the group consisting of
4-{1-Ethyl-6-[(4-fluorophenyl)oxy]-7-phenyl-1H-imidazo[4,5-c]pyridin-2-yl}-furazan-3-amine;
4-{1-Ethyl-7-(4-fluorophenyl)-6-[(4-fluorophenyl)oxy]-1H-imidazo[4,5-c]pyridin-2-yl}-furazan-3-amine;
4-{1-Ethyl-7-{3-[(ethylamino)methyl]phenyl}-6-[(4-fluorophenyl)oxy]-1H-imidazo[4,5-c]pyridin-2-yl}-furazan-3-amine; and
4-{7-{[(3S)-3-Amino-1-pyrrolidinyl]carbonyl}-1-ethyl-6-[(4-fluorophenyl)oxy]-1H-imidazo[4,5-c]pyridin-2-yl}-furazan-3-amine.

4. A method of inhibiting Rho-kinases comprising administering to a subject in need thereof a safe and effective amount of a compound according to claim 1.

5. A method according to claim 4 wherein the disease is selected from the group consisting of:
hypertension, chronic and congestive heart failure, ischemic angina, cardiac hypertrophy and fibrosis, restenosis, chronic renal failure, atherosclerosis, asthma, male erectile dysfunctions, female sexual dysfunction and over-active bladder syndrome, stroke, multiple sclerosis, Alzheimer's disease, Parkinson's disease, amyotrophic lateral sclerosis, inflammatory pain, rheumatoid arthritis, irritable bowel syndrome, inflammatory bowel disease, Crohn's diseases, indications requiring neuronal regeneration, inducing new axonal growth and axonal rewiring across lesions within the CNS, spinal cord injury, acute neuronal injury, Parkinsons disease, Alzheimers disease, cancer, tumor metastasis, viral and bacterial infection, insulin resistance and diabetes.

6. A method according to claim 5 wherein the disease is selected from the group consisting of:
hypertension, chronic and congestive heart failure, ischemic angina, asthma, male erectile dysfunction, female sexual dysfunction, stroke, inflammatory bowel diseases, spinal cord injury, glaucoma and tumor metastasis.

7. A method according to claim 5 wherein the disease is selected from the group consisting of:
hypertension, chronic and congestive heart failure and ischemic angina.

8. A pharmaceutical composition comprising a compound according to claim 1 and a suitable carrier.
